(19) United States
(12) Patent Application Publication
(10) Pub. No.: US 2026/0256358 A1
(43) Pub. Date: Sep. 3, 2026

(54) APPARATUS, METHODS AND SYSTEMS FOR LASER SAFETY INTERLOCK FOR CATHETER

(71) Applicant: Canon U.S.A., Inc., Melville, NY (US)

(72) Inventor: Daisuke Yamada, Cambridge, MA (US)

(21) Appl. No.: 18/993,225

(22) PCT Filed: Jul. 11, 2023
(86) PCT No.: PCT/US2023/069939

§ 371 Date: Jan. 10, 2025

Related U.S. Application Data
(60) Provisional application No. 63/388,145, filed on Jul. 11, 2022.

(51) Int. Cl. A61B 5/00

(52) U.S. Cl. CPC A61B 5/0084; A61B 5/0035; A61B 5/0066; A61B 5/0071; A61B 2562/0233

(57) ABSTRACT

The present patent application aims to teach imaging apparatus, methods, and systems for providing a laser safety interlock for at least one optical probe in the apparatus. The interlock mechanism ensures the lasers are off when the optical probe is not spinning to reduce the laser hazard.

### CROSS REFERENCE TO RELATED PATENT APPLICATIONS

This application claims priority from U.S. Provisional Patent Application No. 63/388,145, filed on Jul. 11, 2022, in the United States Patent and Trademark Office, the disclosure of which is incorporated by reference herein, in its entirety.

### FIELD OF THE DISCLOSURE

The present disclosure relates in general to optical imaging apparatus, methods and systems, and more particularly, to a combined fluorescence and optical coherence tomography catheter having an interlocking system for limiting the laser light generated by the imaging apparatus to reduce the likelihood of injury.

### BACKGROUND OF THE DISCLOSURE

Optical coherence tomography (OCT) provides high-resolution, cross-sectional imaging of tissue microstructure in situ and in real-time, while fluorescence imaging enables visualization of molecular processes. The integration of OCT with fluorescence imaging in a single catheter provides the capability to simultaneously obtain co-localized anatomical and molecular information from the subject tissue, such as an artery wall. For example, in “Ex. Vivo catheter-based imaging of coronary atherosclerosis using multimodality OCT and NIRAF excited at 633 nm” (Biomed Opt Express 2015, 6(4):1363-1375), Wang discloses an OCT-fluorescence imaging system using He:Ne excitation light for fluorescence and swept laser for OCT simultaneously through the optical fiber probe.

In optical imaging systems, a patient interface unit (PIU) is used to interface between the catheter and the system console. The PIU comprises of a fiber optic rotary joint, rotational motor, translational motor and drivers for the motors. In order to acquire cross-sectional images of tubes and cavities such as vessels, esophagus and nasal cavity, the optical probe, which is housed in catheter sheath, is rotated with a fiber optic rotary joint (FORJ) with the rotational motor. In addition, the optical probe is simultaneously translated longitudinally during the rotation with the translational motor so that helical scanning pattern images are obtained. This translation is most commonly performed by pulling the tip of the probe back towards the proximal end and therefore referred to as a pullback. As the system uses a laser beam to illuminate samples, such as tissue, improper use or exposure to the laser light may lead to permanent or temporary damage of the eye, requiring a means for controlling the laser to minimize unwanted exposure.

By way of example, the reference in EP 2698105, titled “Laser Interlock System for Medical Use” to Samsung Electronics Co. LTD, teaches an ultrasound data acquisition unit (210) to acquire ultrasound data on a subject, said ultrasound data acquisition unit comprising an ultrasound probe; a light source unit (220) to generate laser light; and a control unit (230) to turn on or off the light source unit in response to the acquired ultrasound data; wherein the control unit is configured to determine whether or not contact between the subject and the probe occurs using the acquired ultrasound data, and turns on or off the light source unit according to said determination.

The determination is characterized in that the control unit includes: an image generator to generate a 2-Dimensional (2D) ultrasound image using the acquired ultrasound data; a profile detector to detect a profile of the subject from the 2D ultrasound image; and a state determiner to compare the detected profile of the subject with profile sample information corresponding to the subject, so as to calculate a profile difference; wherein the state determiner is configured to determine that contact between the subject and the probe occurs if the calculated profile difference is less than a preset threshold value, and wherein the control unit further includes a light source controller to turn on the light source unit if the state determiner determines that contact between the subject and the probe occurs.

However, this system is lacking in that the optical probe in the catheter is spinning while imaging with the laser active in normal conditions. In this case, the laser beam will only be exposed for a short time when the beam is directed to the eye. In two viewing conditions provided in Table 1 (delta in distance and aperture diameter), the spinning of the optical power will significantly reduce laser exposure in normal conditions, however, this significant reduction in not achieved when the optical probe is stationary (no rotation) while the laser is operating, because of lasers/motor timing control uncertainty and/or software malfunctions. The two viewing conditions in Table 1 below comprise of: 1. A distance of 2000 mm between the eye and catheter laser with an aperture of 50 mm; and 2. A distance of 100 mm with an aperture of 7 mm. The optical power to the aperture will reduce to 0.4% and 1.1%, respectively, shown in Table 1, when the optical probe is spinning compared to the optical power when the optical probe is stationary (not spinning and aim to the aperture). The spinning of the optical power will significantly reduce laser exposer at normal condition, however, if the optical probe is stationary (no rotation) while the laser is on because of lasers/motor timing control uncertainty and/or software malfunctions, the reduction is insignificant.

TABLE 1

| | Viewing | |
|---|---|---|
| | Conditions | |
| | 1 | 2 |
| Distance [mm] | 2000 | 100 |
| Aperture diameter [mm] | 50 | 7 |
| Optical Power reduction | 0.40% | 1.11% |

Accordingly, and in view of the above-referenced issues, the present innovation provides apparatus, methods and systems for alleviating shortcomings in the established art.

### SUMMARY

The present patent application aims to teach apparatus, methods, and systems for eliminating or significantly reducing laser exposure in an optical probe.

In one embodiment, the subject disclosure teaches an optical imaging apparatus comprising: an imaging engine having at least one laser source and a laser controller; a patient interface unit having a rotational motor, a rotary joint, and a probe connection; and a probe comprising an optical fiber to illuminate a laser light from the at least one laser source; wherein the probe is rotated by the rotational motor when imaging; and wherein the laser light is functional only when the probe is rotating.

In additional embodiment the subject optical imaging apparatus also comprising an interlock circuit for controlling functionality of the laser light.

In yet additional embodiment, the interlock circuit further comprises a frequency comparator to generate the laser light on and off states depending on the rotating speed of the rotational motor. It is further contemplated that the interlock circuit is a redundancy circuit, wherein the redundancy circuit includes 2 frequency comparators, wherein one comparator is a positive comparator, and the other comparator is negative comparator.

In another embodiment, the laser light is functional only when the probe is rotating above a threshold. Furthermore, the threshold may be greater than or equal to 1000 revolutions per minute.

In yet another embodiment of the subject optical imaging apparatus, the rotational motor has an encoder to generate spinning signals for calculating rotating speed. Furthermore, the spinning signal from the encoder is greater than or equal to 100 pulses per rotation.

In further embodiment, the subject disclosure teaches an optical imaging method having a optical imaging apparatus comprising: an imaging engine having at least one laser source and a laser controller; a patient interface unit having a rotational motor, a rotary joint, and a probe connection; and a probe comprising an optical fiber to illuminate a laser light from the at least one laser source; wherein the probe is rotated by the rotational motor when imaging; and wherein the laser light is functional only when the probe is rotating.

### BRIEF DESCRIPTION OF THE DRAWINGS

Further objects, features and advantages of the present disclosure will become apparent from the following detailed description when taken in conjunction with the accompanying figures showing illustrative embodiments of the present invention.

FIG. 1 is a schematic illustration of an exemplary OCT-fluorescence imaging system according to one or more embodiment of the subject innovation.

FIG. 2 is a system overview of an exemplary OCT-fluorescence imaging system according to one or more embodiment of the subject innovation.

FIG. 3 provides a schematic illustration of an exemplary OCT-fluorescence multi-modality imaging system, according to one or more embodiment of the subject innovation.

FIG. 4 is a block diagram depicting an exemplary OCT-fluorescence multi-modality imaging system, according to one or more embodiment of the subject innovation.

FIG. 5 provides a overview of an exemplary free space beam combiner in the PIU, according to one or more embodiment of the subject apparatus, method or system.

FIG. 6 is side view of an exemplary catheter for OCT-fluorescence multi-modality imaging system, according to one or more embodiment of the subject apparatus, method or system.

FIG. 7 provides an exemplary architecture for a laser safety interlock, according to one or more embodiment of the subject apparatus, method or system.

FIG. 8 is a diagram depicting an exemplary frequency comparators in an OCT-fluorescence multi-modality imaging system, according to one or more embodiment of the subject apparatus, method or system.

FIG. 9 is a diagram/flow chart detailing an exemplary OCT-fluorescence multi-modality imaging system, according to one or more embodiment of the subject apparatus, method or system.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

The fiber optic catheters and endoscopes have been developed to access to internal organs. For example in the cardiology, OCT (optical coherence tomography), white light back-reflection, NIRS (near infrared spectroscopy) and fluorescence technology have been developed to see structural and/or molecular images of vessels with a catheter. The catheter, which comprises a sheath and an optical probe, is navigated to a coronary artery.

In order to acquire cross-sectional images of tubes and cavities such as vessels, esophagus and nasal cavity, the optical probe is rotated with a fiber optic rotary joint (FORJ). In addition, the optical probe is simultaneously translated longitudinally during the rotation so that helical scanning pattern images are obtained. This translation is most commonly performed by pulling the tip of the probe back towards proximal end and therefore referred to as a pullback.

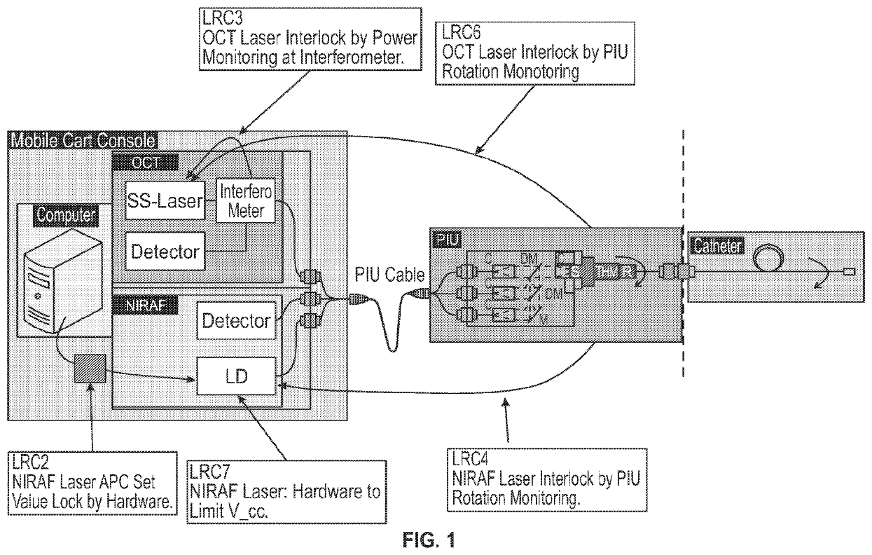

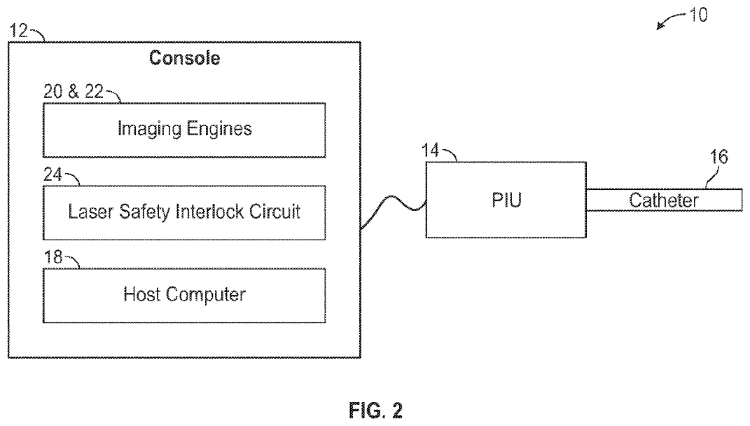

Imaging of coronary arteries by exemplary intravascular OCT and fluorescence systems is described in the 1^{st} embodiment, provided in FIGS. 1 and 2. In this embodiment, the system 10 provides a laser safety interlock to ensure the lasers are off when the optical probe is not spinning.

In detailing the system overview, the imaging system 10 includes a console 12, a PIU 14 (patient interface unit) and a catheter 16. The console 12 comprises a host computer 18, imaging engines (OCT engine 20 and fluorescence engine 22) and laser safety interlock circuits 24. The OCT and fluorescence laser sources 26 and 50 and the controller 62 are housed in the imaging engines 20 & 22, and the laser safety interlock circuits 24 provide the interlock signals to the laser controller 62 to ensure that both lasers are on only when a rotational motor 58 in the PIU 14 is spinning. The detailed description of each component and their interactions is explained in the following section.

### OCT Engine

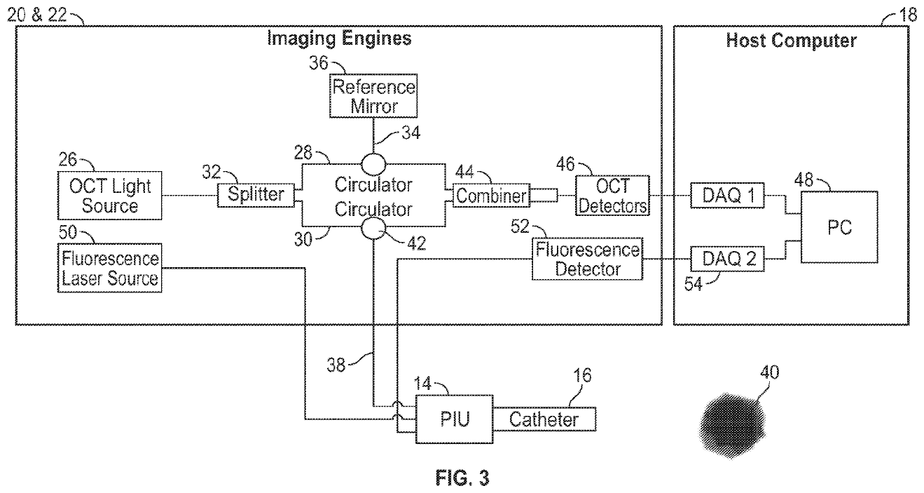

With reference to FIG. 3, an OCT laser beam with a wavelength of around 1.3 um from an OCT light source 26 is delivered and split into a reference arm 28 and a sample arm 30 with a splitter 32. A reference beam 34 is reflected from a reference mirror 36 in the reference arm 28 while a sample beam 38 is reflected and/or scattered from a sample 40 through a PIU 14 (patient interface unit) and a catheter 16 in the sample arm 30. Fibers of the PIU 14 and catheter are made of a DCF (double clad fiber). The OCT laser beam illuminates the sample 40 (outside of the catheter) through the core of DCF, and scattered light from the sample 40 is collected and delivered back to the circulator 42 of an OCT interferometer via the PIU 14 and combined with reference beam 34 at the combiner 44 and generate interference patterns. The output of the interferometer is detected with the OCT detectors 46 such as photodiodes or multi-array cameras. Then signals are transferred to a processor 48 to perform signal processing to generate OCT images. The interference patterns are generated only when the path length of the sample arm matches that of the reference arm to within the coherence length of the light source.

### Fluorescence Engine

An excitation laser with wavelength of 0.635 um from a fluorescence light source 50 delivers to the sample 40 (outside of the catheter) through the PIU 14 and the catheter 16. The patient interface unit (PIU, explained in more detail below) comprises a free space beam combiner so that the excitation light couples into the common DCF with OCT.

The excitation laser 26 and/or 50 illuminates the sample 40 from the distal end of the optical probe in the catheter 16. The sample 40 emits auto-fluorescence with broadband wavelengths of 0.65-0.90 um. The auto-fluorescence is delivered to a fluorescence detector 52 such as photo-multi plier tube (PMT) via the PIU 14. Then, the analog electrical signal at the fluorescence detector 52 is acquired by a data acquisition board (DAQ 2) 54.

### PIU (Patient Interface Unit)

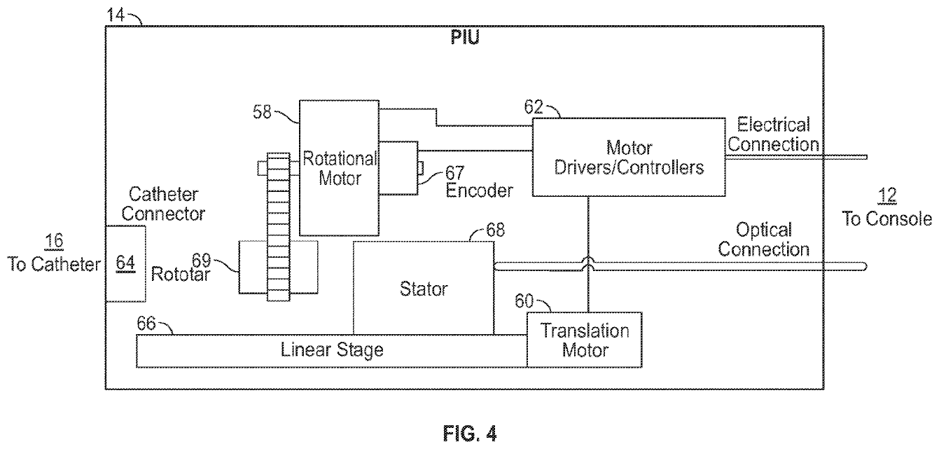

The PIU 14 is interfaced between the catheter 16 and the console 12, and the PIU 14 provides the means to spin and linearly translate the catheter's imaging core (optical probe) within the catheter's outer sheath. The PIU 14 comprises a free space beam combiner, a FORJ 56 (Fiber Optic Rotary Joint), rotational motor 58 and translation motor 60 and linear stage 66, the motor drivers/controllers 62, and a catheter connector 64, as can be seen in FIG. 4.

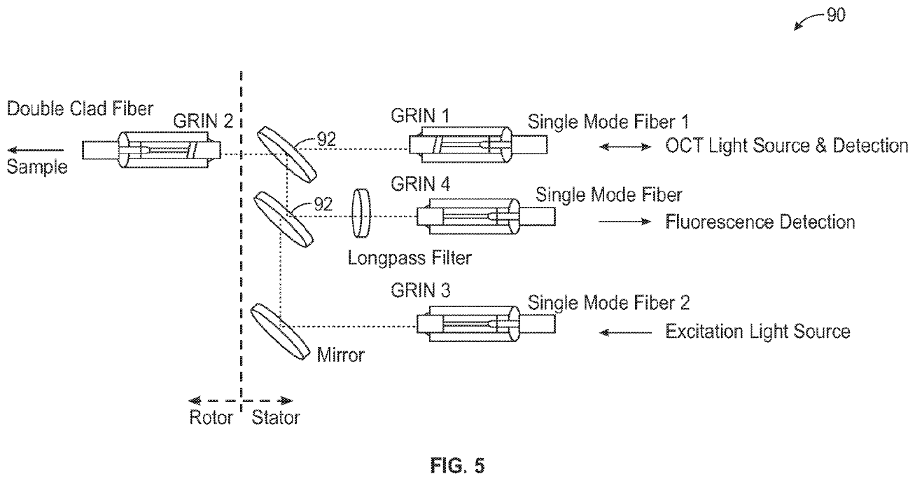

The FORJ 56 (shown in greater detail in FIG. 5) allows uninterrupted transmission of an optical signal while rotating the double clad fiber on the left side along the fiber axis in FIG. 5. The FORJ 56 has a free space optical beam coupler to separate a rotator 69 and a stator 68. The rotator 69 comprises a double clad fiber with a lens to make collimated beam. The rotator 69 is connected to the optical probe 16, and the stator 68 is connected to the optical sub-systems.

The free space beam combiner 90 has dichroic filters 92 to separate different wavelength lights (OCT laser, excitation laser and auto-fluorescence lights). The beam combiner also comprises low-pass filters or band-pass filters in front of the auto-fluorescence channel to eliminate excitation light to minimize excitation light noises at the fluorescence detector. The cut-of wavelength of the filter (low-pass or band-pass) is selected around from 645 to 700 nm.

The rotational motor 58 delivers the torque to the rotor. Also, the translation motor 60 and linear stage 66 is used for a pullback, and motor drivers/controllers 62 (hereafter referenced as Controller or Motor Driver) drive both rotational motor 58 and translation motor 60. An encoder 67 is attached to the rotational motor 58 to generate encoder signal outputs for feedback to the rotational motor 58 and also to provide the signals to the laser safety interlock circuits 24 to monitor the rotational motor 58 movements.

### Catheter

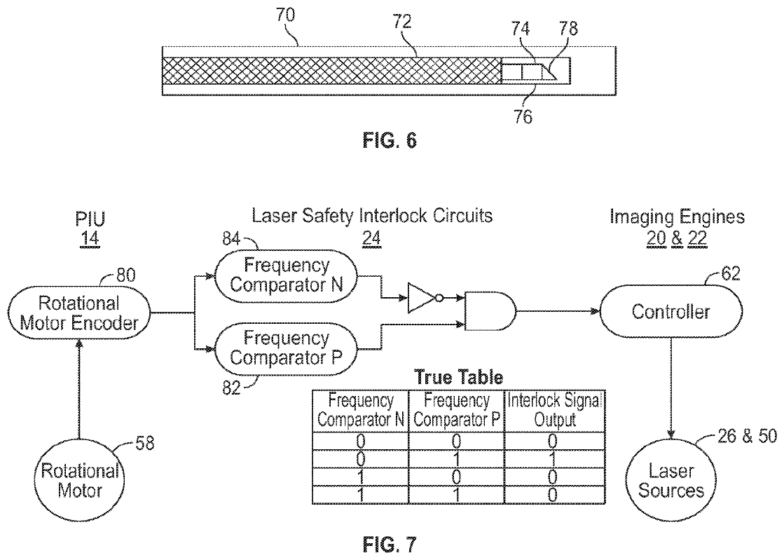

The catheter 16, depicted in FIG. 6, includes a sheath 70, a coil 72, a protector 76 and an optical probe 74. The catheter 16 is connected to the PIU 14. The optical probe 74 comprises an optical fiber connector, an optical fiber and a distal lens 78. The optical fiber connector is used to physically engage with the PIU 14. The optical fiber delivers light to the distal lens 78, and the distal lens 78 is to shape the optical beam and to illuminate light to the sample 40, and to collect light from the sample 40 efficiently.

The coil 72 delivers the torque from the proximal end to the distal end by the rotational motor 58 in the PIU 14. There is a mirror at the distal end of the catheter, so that the light beam is deflected outward. The coil 72 is fixed with the optical probe 74 so that a distal tip of the optical probe 74 also spins to see omnidirectional view of the inner surface of hollow organs such as vessels. The optical probe 74 comprises a fiber connector at proximal end, double clad fiber and a distal lens 78 at distal end. The fiber connector is connected with the PIU 14. The double clad fiber is used to transmit & collect OCT light through the core and to collect auto-fluorescence from samples 40 through the clad. The distal lens 78 is used for focusing and collecting light to and/or from the sample. The scattered light through the clad is relatively higher than that through the core because of the size of the core is much smaller than the clad.

### Laser Safety Interlock

The laser safety interlock circuit 24 provides the laser safety interlock signal to the laser controller 62. FIG. 7 details the high level architecture associated with the safety interlock circuit 24. The rotational motor 58 encoder 67 signals from the PIU are delivered to frequency comparators 82 and 84 in the laser safety interlock circuit 24. The frequency comparators 82 and 84 monitor and generate the digital signal when exceeding a threshold. The two frequency comparator 82 and 84 circuits (Frequency Comparator P 82, Frequency Comparator N 84) add redundancy and the two signals being logic opposites of each other helps with detecting any transmission issues between the laser safety interlock circuits 24 to imaging laser controllers 62.

The frequency comparator P 82 signal output is logic HIGH when the rotation speed is above the threshold and logic LOW otherwise. The frequency comparator N 84 signal output is logic LOW when the rotation speed is above the threshold and logic HIGH otherwise. The two output signals from the frequency comparators are fed into the logic circuits to generate only when valid states of frequency comparators. The true table is shown in FIG. 7. Then, the interlock signal is delivered to the laser controller 62, and the laser controller 62 shut-down the laser.

By way of example, the threshold is set at 1000 rpm, so that a maximum duration of laser emission to an aperture of 7 mm at 100 mm distance will be

$$\frac{60}{1000} \times \frac{0.07}{2 π} = 0.6 \; 68 ( msec ) .$$

Also, the response time of less than 0.250 msec is set, so that the circuits shut down the laser immediately. Note, the response time is defined as the motor spinning speed behind the threshold until laser emissions are shut down.

In a worst case scenario, the rotational motor suddenly stops just after the duration period of 1000 rpm, the time basis will be 0.668+0.250=0.918 msec. The threshold and the response time are determined based on the optical power and the time basis that the system allows to expose the laser beams, but in general, higher threshold value and faster response time to help minimize laser exposure.

Hall sensor signals can be used instead of the encoder signals to represent the rotational motor movements, so that the rotational motor will be simplified in not having the encoder 67 to achieve laser safety interlock circuit inputs. However, the encoder 67 generates multiple pulses per rotation (e.g. 500 pulses per rotation) and this helps to reduce the response time. The frequency comparator 82 and 84 requires at least a few pulses to determine the pulse frequency, and when the threshold is 1000 rpm, the pulse period is 60 msec. When the encoder signals 100 pulses per rotation, the pulse period will be 0.60 msec. When the encoder 67 signals is more than 100 pulses per rotation, the time base becomes faster, and thus that will help to minimize laser exposure to the end users.

### Frequency Comparator

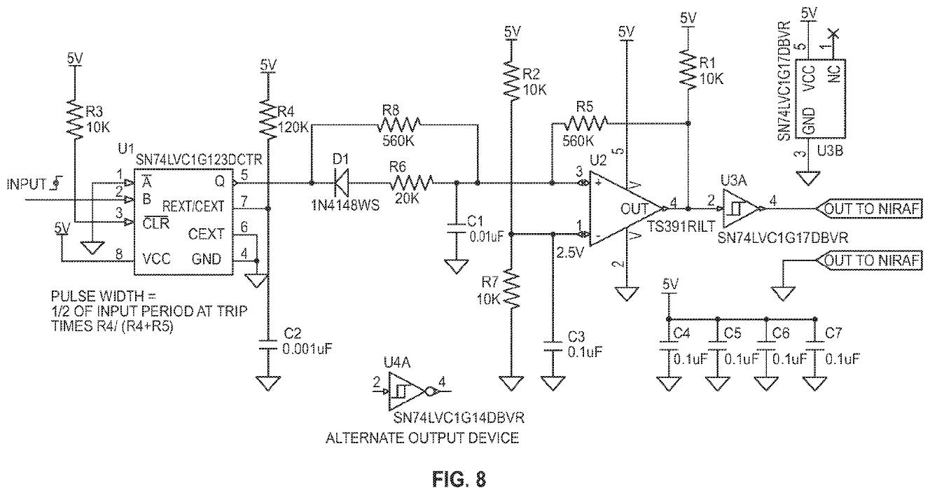

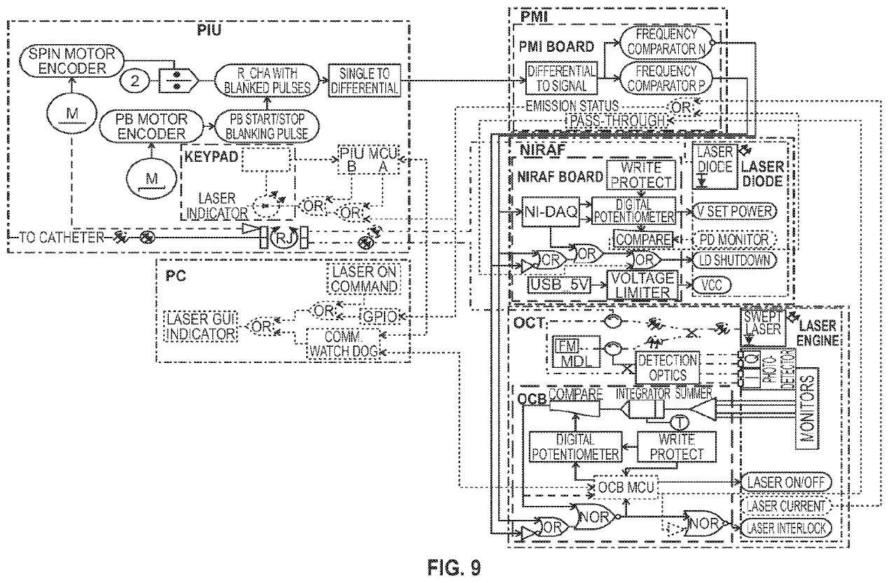

FIG. 8 describes the circuit to achieve a fast response time. The signal to be detected, a digital pulse-train from the PIU Spin Encoder, is applied to the B-input of the re-triggerable multivibrator, U1. The output pulse-width of U1 is set by a resistor, R4, and capacitor, C2, to a pulse period equal to the period of the desired trip frequency.

When the motor is not spinning U1 is not triggered, its' output is low, and the voltage on capacitor C1 is zero. The output of U2 and U3 are low and the NIRAF is not enabled. When the motor spins, U1 is triggered by each rising-edge of the pulse-train, and C1 charges. When the pulse-train frequency exceeds the minimum acceptable frequency C1 charges to VCC. U2 trips at ½ VCC enabling the NIRAF laser. Resistor R5 provides hysteresis so that once triggered the pulse-train frequency must decrease to remove the enable signal to the NIRAF. As the frequency of the pulse-train is reduced to the set point, the voltage on capacitor C1 decreases to the trip point, the comparator goes low, disabling the NIRAF laser.

In another embodiment, when the motor spins, U1 is triggered by each rising-edge of the pulse-train. The two flip-flops are also triggered on the rising-edge of the pulse-train. When the pulse-period of U1 is less than the trigger frequency, the flip-flops clock a logic-o (clocking before the U1 output has gone from low to high) and the laser is disabled. When the pulse-train frequency exceeds the minimum acceptable frequency, U1 retriggers and the output is a continuous high. The next rising edge of the pulse-train clocks a logic-1 into U2A, and the second rising edge of the pulse-train clocks a logic-1 into U2B, and the laser is enabled.

1. Optical imaging apparatus comprising:
an imaging engine having at least one laser source and a controller for controlling the laser source;
a patient interface unit comprising:
a rotational motor;
rotary joint; and
a probe connection; and
a probe comprising an optical fiber to illuminate a laser light from the at least one laser source;
wherein the probe is rotated by the rotational motor when imaging; and
wherein the laser light is functional only when the probe is rotating.

2. The apparatus of claim 1, further comprising an interlock circuit for controlling functionality of the laser light.

3. The apparatus of claim 2, wherein the interlock circuit further comprises a frequency comparator to generate the laser light on and off states depending on the rotating speed of the rotational motor.

4. The apparatus of claim 2, wherein the interlock circuit is a redundancy circuit.

5. The apparatus of claim 4, wherein the redundancy circuit includes 2 frequency comparators, wherein one comparator is a positive comparator, and the other comparator is negative comparator.

6. The apparatus of claim 1, wherein the laser light is functional only when the probe is rotating above a threshold.

7. The apparatus of claim 4, wherein the threshold is greater than or equal to 1000 revolutions per minute.

8. The apparatus of claim 1, wherein the rotational motor has an encoder to generate spinning signals for calculating rotating speed.

9. The apparatus of claim 8, wherein the spinning signal from the encoder is greater than or equal to 100 pulses per rotation.

10. A method for operating an optical imaging apparatus, the optical imaging apparatus comprising:
an imaging engine having at least one laser source and a controller for controlling the laser source;
a patient interface unit comprising:
a rotational motor;
rotary joint; and
a probe connection; and
a probe comprising an optical fiber to illuminate a laser light from the at least one laser source;
the method comprising:
rotating the probe using the rotational motor;
illuminating the laser light when the probe is rotating to capture an image;
terminating the laser light once a disturbance of the probe occurs.

11. The method of claim 10, further comprising an interlock circuit for controlling functionality of the laser light.

12. The method of claim 10, wherein the disturbance of the probe occurs when a frequency comparator to generate the laser light on and off states, which depends on the rotating speed of the rotational motor, reaches or exceeds a threshold.

13. The method of claim 11, wherein the interlock circuit is a redundancy circuit.

14. The method of claim 13, wherein the redundancy circuit includes 2 frequency comparators, wherein one comparator is a positive comparator, and the other comparator is negative comparator.

15. The method of claim 10, wherein the laser light is functional only when the probe is rotating above a threshold.

16. The method of claim 13, wherein the threshold is greater than or equal to 1000 revolutions per minute.

17. The method of claim 10, further comprising generating a spinning signal from an encoder in the rotational motor for calculating rotating speed.

18. The method of claim 17, wherein the spinning signal from the encoder is greater than or equal to 100 pulses per rotation.
